(19) United States
(12) Patent Application Publication
(10) Pub. No.: US 2006/0222642 A1
(43) Pub. Date: Oct. 5, 2006

(54) Compositions for the treatment of mycobacterial infections

(76) Inventors: Armando Acosta Dominguez, Habana (CU); Armando Cadiz Lahenz, Habana (CU); Gustavo Falero Diaz, Habana (CU); Victoriano Gustavo Sierra Gozalez, Habana (CU); Maximo Berto Martinez Benitez, Habana (CU); Maria Elena Sarmiento Garcia San Miguel, Habana (CU); Yamile Lopez Hernandez, Habana (CU); Juan Francisco Infante Bourzac, Habana (CU)

Correspondence Address: LACKENBACH SIEGEL, LLP, LACKENBACH SIEGEL BUILDING, 1 CHASE ROAD, SCARSDALE, NY, 10583, US

(21) Appl. No.: 10/536,298

(22) PCT Filed: Nov. 25, 2003
(86) PCT No.: PCT/CU03/00015

§ 371 Date: Apr. 17, 2006

(30) Foreign Application Priority Data
Nov. 25, 2002 (CU) 2002-0275

(51) Int. Cl. A61K 39/395; A61K 39/40

(52) U.S. Cl. 424/130.1; 424/168.1

(57) ABSTRACT

The invention relates to the field of immunology and, more specifically, to the treatment of infectious diseases caused by mycobacteria, whereby antibody compositions are administered mucosally and in other ways for the prophylactic and therapeutic treatment of said diseases. The invention relates to the preparation of compositions of human antibodies of class IgG, secretory IgA or mixtures of both having a specific activity against mycobacterial antigens for the prevention and treatment of infections caused by said type of micro-organism. The inventive compositions have demonstrated their ability to prevent in vivo mycobacterial infection.

The present invention has applications in the field of immunology—specifically in the control of infectious diseases caused by mycobacteria—based on the use of preparations containing antibodies, administered through mucous membranes and other routes, which have a prophylactic and therapeutic function.

Among existing mycobacterial organisms, we find pathogens which prove dangerous to both animals and man, including the Mycobacterium tuberculosis, which causes tuberculosis, Mycobacterium leprae, responsible for leprosy, Mycobacterium avium and Mycobacterium intracelulare, which bring about tuberculosis in immunodepressed patients, as well as other mycobacteria which cause diseases in human beings with far less frequency (Sommer H M, Good R C. Mycobacterium. In: Manual of clinical Microbiology, 4^{th} Ed. Washington D.C.: A society for Microbiology; 1985. p. 216-248., Orme I M. Immunity to mycobacteria. Current Opinion in Immunology. 1993; 5: 497-502)

In the case of animals, the Mycobacterium avium, subspecies pararatuberculosis, which causes Jones Disease in ruminants, and Mycobacterium bovis, which causes tuberculosis in cattle, are especially noteworthy (Dannenberg Am. Pathogenesis of Tuberculosis: native and acquired resistance in animals and humans. In: Leive L, Schlesinger D (eds). Microbiology. 1984, p. 344-354).

Among the most important mycobacterial diseases affecting man, the tuberculosis (TB) infection constitutes a health concern for the entire world and is the primary cause of death associated with infectious diseases, despite the use of the BCG vaccine and of a great number of drugs designed to control it (Dolin P J, Raviglione M K, Kochi A. Global tuberculosis, incidence and mortality during 1990-2000. Bull Who. 2001; 72: 213).

It is estimated that one third of the world's population has been infected by the Mycobacterium tuberculosis. Every year, 8 million people throughout the world develop active TB and 3 million die from it. A co-infection with the Human Immune-deficiency Virus (HIV), represents 3 to 5 percent of cases (Dolin P J, Raviglione M K, Kochi A. Global tuberculosis, incidence and mortality during 1990-2000. Bull Who. 1994; 72: 213).

The considerable spread of the disease has necessitated the development of new and better diagnostic methods, as well as that of vaccines and therapeutic agents (Collins F M. Tuberculosis: The Return of an Old Enemy. Critical Reviews in Microbiology. 1993; 19: 1-16).

With respect to treatment, this has consisted in a combination of medicines administered in relatively high doses and for extended periods of time, something which leads to an associated toxicity that renders difficult the implementation of controlled treatment programs (McCarthy M. Experts see progress in fight against tuberculosis. Lancet. 2002; 359: 2005). In this regard, it would be desirable to shorten the treatment period, to thus favor the application and the completion of a control program, which would prevent the appearance of resistant strains. A reduction in the dosage of the drugs administered would also prove useful in reducing the toxicity of the overall treatment.

The appearance of strains with multiple resistance to drugs and the great many patients that carry them, as well as the growing number of cases that promise to make an appearance in the future, is a growing concern that demands the development of new therapeutic alternatives (McCarthy M. Experts see progress in fight against tuberculosis. Lancet. 2002; 359: 2005; Hopwell P C. Tuberculosis Control: How the world has changed since 1990. Bull. World Health Org 2002, 80:427, Freire M, Rosigno G. Joining forces to develop weapons against TB: together we must. Bull. World Health Org 2002, 80:429).

In addition to this, there exist many species of mycobacteria that produce diseases in man for which there is no adequate treatment.

The BCG is the one, existing vaccine used in humans. In the entire world, a total of 3×10¹² doses have been administered. Its efficacy varies greatly depending on the strain employed—its nutritional state, its genetic background, the aging process it has undergone and the presence of intercurrent infections. Its is considered efficacious only in the prevention of serious forms of the illness (the miliary and meningeal forms) affecting infants, and to be useless in the prevention of pulmonary tuberculosis, a fact urgently requiring the development of new vaccines (Hirsch L S, Johnson-J L, Eliner J J. Pulmonary tuberculosis. Curr-Opin-Pulm-Med 1999; 5(3):143-50; Jacobs G G, Johnson J L, Wallis R S. Tuberculosis vaccines: how close to human testing. Tuber Lung Did 1997; 78:159-169; Ginsberg A M. What's new in tuberculosis vaccines? Bull. World Health Org 2002, 80:483).

The decisive role of cell-mediated immunological responses, fundamentally by the TH1 sub-population, in the fight against Mycobacterium tuberculosis, and against mycobacteria in general, has already been recognized and constitutes an international dogma which has the infection as inevitable (Specificity and Function of T- and B-cell Recognition in Tuberculosis: Pathogenesis, Protection and Control. J infect. Dis. 1994; 30: 117-135).

Following this reasoning, all of the vaccination strategies utilized in the development of new vaccines for use in the fight against tuberculosis and other mycobacteria aim at stimulating this immune response mechanism (Ginsberg A M. What's new in tuberculosis vaccines? Bull. World Health Org 2002, 80:483). However, the role that specific antibodies may play in defense against mycobacteria has not been studied extensively (Glatman A. Casadevall A. Serum therapy for tuberculosis revisited: reappraisal of the role of antibody mediate immunity against Mycobacterium tuberculosis. Clin Microbiol Rev. 1998; 11:514-532).

Mycobacterial organisms penetrate the body through the mucosa; this means that the presence of specific antibodies in secretions could constitute a barrier to the penetration of these microorganisms, preventing the infection. Moreover, the arrival of specific antibodies from the mucosa, the blood or other parts of the organism at the site of an existing infection could have therapeutic effects.

To date, the therapeutic work carried out on animals and man involving the use of serums and preparations containing polyclonal antibodies administered by injection, has not yielded conclusive results (Glatman A. Casadevall A. Serum therapy for tuberculosis revisited: reappraisal of the role of antibody mediate immunity against Mycobacterium tuberculosis. Clin Microbiol Rev. 1998; 11: 514-532).

In the case of studies conducted on human beings, carried out at the close of the nineteenth century and the beginning of the twentieth, the serums employed were always obtained from other species, with few details given about their form of extraction, their strength, etc., and the investigations carried out lacked the methodological and statistical rigor that would allow for valid conclusions to be reached (Glatman A. Casadevall A. Serum therapy for tuberculosis revisited: reappraisal of the role of antibody mediate immunity against Mycobacterium tuberculosis. Clin Microbiol Rev. 1998; 11: 514-532).

Pre-incubation of Mycobacterium tuberculosis with a monoclonal antibody, antilipoarabinomannan, before administering the microorganism to mice in aerosol form, prolonged the survival rate in comparison to the group of untreated animals, though it did not reduce the number of microorganisms in the organs studied (Teitelbaum R. Glatman-Freedman A, Chen B, Robbins J B, Unanue E, Casadevall A, Bloom B. A mAb recognizing a surface antigen of Mycobacterium tuberculosis enhances host survival. Proc Natl Acad Sci USA. 1998; 95: 15688-15693; US Patent Application 20010007660). It is noteworthy that when the effect of administering the antibody “in vivo” through the peritoneum was evaluated, no beneficial effects were detected.

One of the chief disadvantages of the aforementioned strategies is the use of serums and preparations containing antibodies that are obtained from other species, in which antigenic recognition is different, as are the classes and sub-classes of antibodies which—being from species other than man—do not take advantage of all the antibody-dependent, immune effector mechanisms of the human being, greatly reducing their effectiveness. Another disadvantage is found in concomitantly administering heterologous proteins, which leads to blood disease, a serious complication with systemic repercussions, especially for joints and kidneys, together with inactivation of the antibodies due to the response of anti-species antibodies generated by the receptor, leading to the loss of anti-microbial activity in the preparation employed.

In addition to these drawbacks, monoclonal antibodies have the disadvantage of limiting their action to only one epitope of one of the microorganism's antigens, which diminishes their anti-microbial potential and the possibility of employing them against different kinds of mycobacteria. The combination of these elements make these strategies inefficient and dangerous in practice.

The aim of the invention presented is the development of preparations containing human antibodies, alone or in combination with immuno-potentiators, immuno-modulators, inactivated or attenuated strains, antigens, genes and/or drugs to be administered through the mucous membrane, as well as other routes, to prevent or treat infections brought on by mycobacteria.

The preparations of antibodies employed are made up of:
IgG human antibodies obtained through any of the known, state-of-the-art methods, such as alcoholic precipitation, ion-interchange chromatography, and a stage of viral inactivation.
IgA secretory and lactoferrous human antibodies, obtained by means of colostrum through any of the known, state-of-the-art methods.
A mixture of the mentioned preparations

All of the preparations were obtained from a large number of individuals, and prior to their employment, the presence of high concentrations of antibodies specific to antigens of Mycobacterium tuberculosis and BCG, were detected in the samples. Surprisingly, a wide range of activity against mycobacterial antigens was achieved, allowing for the use of this type of preparation in the prevention and treatment of a broad spectrum of infections produced by this kind of microorganism, bearing in mind the wide, cross reactivity of the antigens among the components of the bacterial species.

These preparations may be used for prophylaxis in groups at risk of mycobacterial infection, such as those who come in contact with patients (including relatives, friends, professionals, etc.), or among those patients who are especially predisposed to contracting these infections, as is the case of HIV carriers, who are more susceptible to mycobacterial infections, including those produced by Mycobacterium tuberculosis, and especially by Mycobacterium avium and Mycobacterium intracelulare, which penetrate the organism via the gastrointestinal system through the ingestion of food, are resistant to treatment and constitute one of the main causes of death in this group of patients.

On the other hand, these preparations have therapeutic value in the treatment of established infections, such that administering the preparations of antibodies could have a therapeutic effect as well as reducing the treatment period in patients infected with sensitive strains—something of great importance, since the current treatments are lengthy, proving a nuisance to patients and encumbering the work of the public health system; the toxicity of the treatment is increased as is the risk of its not being correctly followed, which can result in the appearance of strains resistant to pharmacological agents, or its application among patients carrying strains with multiple resistance to specific medicines, a growing worldwide phenomenon that currently has no therapeutic alternative.

The preparations representing the present invention produced a significant reduction in levels of BCG pulmonary infections in an intra-nasal model employing mice (example 2). In the preparation of IgG antibodies, a prophylactic and therapeutic effect was also obtained after their administration through the peritoneum. In all of the methods for administering the antibodies, the effective passage of the antibodies to bronchial and salivary secretions has been demonstrated. The inhibition of pulmonary infection was achieved following pre-incubation of the microorganism with the preparations employed (example 2).

The preparations of human gammaglobulin obtained from plasma or colostrum indicated the presence of antibodies that target complete BCG cells and antigens of Mycobacterium tuberculosis (example 1); this implies that the resulting immunological effect cannot be linked to antibodies targeting one antigen in particular, and that the simultaneous recognition of numerous different antigens is a positive quality of these types of preparations, stemming from the antibacterial activity of each kind of specific antibody present in the preparation.

Another objective of the invention presented is the use of the preparations mentioned for prophylaxis and treatment of infections in humans caused by any kind of mycobacteria, including those causing tuberculosis, leprosy and others, as well as their administration via mucous membranes, including injection and intra-nasal methods.

The present invention also relates to preparations additionally containing immuno-potentiators and/or immuno-modulators of any kind: natural, recombining, synthetic, etc., as well as attenuated or inactivated strains, or those containing antigens or their equivalents, or drugs known to be active against mycobacteria.

The present invention offers a novel method for treating and preventing diseases caused by mycobacteria, through the employment of preparations containing IgG and IgA class human antibodies “in vivo”. The employment of IgA secretory antibodies is especially novel, being unmentioned by other authors to date. It is equally novel that said preparations proved effective when administered both systemically and through the mucosa.

The present invention demonstrates, through the results obtained, how human antibodies play a role both in the prevention and in the treatment “in vivo” of a mycobacterial infections, a surprising fact given that antibodies are not described as inhibitors of mycobacterial infections in state-of-the-art techniques, and only cell-mediated protection mechanisms are considered relevant.

The present invention will be described through the following concrete examples:

### EXAMPLE 1

### Distribution Kinetics of Gammaglobulins

Male mice from an isogenic Balb/c pool, ranging from 8 to 10 weeks in age and from 20 to 22 grams in weight, supplied by the National Centre for the Production of Laboratory Animals (CENPALAB), Havana, Cuba, were employed in the experiment. They were inoculated with the Japanese (Tokyo) BCG strain, produced by the BCG laboratory of Japan, and with preparations of human gammaglobulin, obtained through methods described by Cadiz and co-workers (Cadiz A. Fernandez J. Joo L. Moya A. Modifications to the alcoholic fractioning method employed in Cuba for the production of immunoglobulines for intravenous use. Vaccimotor 1998; 7: 2-7). As shown in Table 1, the animals in Groups I and II were administered human gammaglobulin (IgG) intra-peritoneously and intra-nasally respectively, and samples of serum, saliva and tracheobronchial secretion were serially extracted from four different animals belonging to each of the groups. A similar experiment was carried out with animals that received the preparation of IgA antibodies intra-nasally.

TABLE 1
Design of the experiment of human gammaglobulin
distribution kinetics in Balb/c mice.

| | | | | Extraction |
|---|---|---|---|---|
| | Inoculation | Dose of | | Time |
| Groups | Method | Gammaglobulin | Samples | (hours) |
| I | Intra- | 1 mg/g of | Serum. Saliva. | 1, 2, 3 |
| | peritoneous | weight | Tracheobronchial | and 5 |
| | | | secretion. | |
| II | Intra- | 1 mg | Serum. Saliva. | 1, 2, 3 |
| | nasal (IN) | contained | Tracheobronchial | and 5 |
| | | in 20 μL | secretion | |

Each sample indicated the presence of antibodies directed against antigens of Mycobacterium bovis following an ELISA test of complete cells. Antibodies against BCG surface antigens were detected in the preparation of human gammaglobulin IgG with a titer of 4096. In the case of the preparation containing IgA antibodies, specific antibodies were also detected. In Group I, the samples of serum, extracted in the first period (1 hour), had antibodies with a titer of 5120, which increased to 20480 after 2 hours, decreased to 2560 after 3 hours, and remained stable until the end of the experiment (5 hours). In the case of the saliva samples, the results were similar, with titers of 20, 1280, 0 and 0. The presence of antibodies in the tracheobronchial secretions was not detected until 2 hours had elapsed, with a titer of 80, which decreased to 40 after 3 hours, to reach a value of 20 at the end of the experiment (5 hours). As can be seen, the maximum values for the titers of antibodies corresponding to the samples of serum, saliva and tracheobronchial secretion, were those of 20480, 1280 and 80 respectively. These results are shown in FIG. 1.

In the animals belonging to Group II that were intra-nasally inoculated with the preparation of IgG gammaglobulin (FIG. 2), the presence of specific antibodies was not detected in the blood at the different times of the experiment, though antibodies with a titer of 160 were detected in the tracheobronchial secretion samples that were taken during the first hour of the experiment. Upon comparing the results with respect to the inoculation methods, it was observed that the blood samples indicated the presence of specific antibodies only when the gammaglobulin was inoculated intra-peritoneously. In the saliva samples, greater titer values were observed when the inoculation was done intra-peritoneously, a result related to the high levels of antibodies present in the blood of these animals. In the tracheobronchial secretion, the larger titer values for antibodies were obtained when the inoculation was done intra-nasally. The study demonstrates that with both administration methods, antibodies that recognize the BCG gathered in the mucosa of these animals.

In the case of the preparation of IgA antibodies administered intra-nasally, its pattern of distribution was similar to that obtained with the preparation of IgG administered with the same method.

### EXAMPLE 2

### Challenge Experiment

Using the preparations of IgG and IgA secretory antibodies, a challenge experiment was carried out. The design used is shown in Table 3.

TABLE 3
Design of the challenge experiment using BCG in Balb/c mice.

| | Treatment using human | | Inoculation after | |
|---|---|---|---|---|
| | gammaglobulin | | two hours | |
| Groups | Dose | Method | Dose | Method |
| I | — | — | BCG, | IN |
| | | | 0.5 × 10⁶ cells | |
| II | 1 mg/g of weight | IP | BCG, | IN |
| | | | 0.5 × 10⁶ cells | |
| III | 1 mg contained in | IN | BCG, | IN |
| | 20 μL. | | 0.5 × 10⁶ cells | |
| IV | — | — | 0.5 × 10⁶ cells | IN |
| | | | pre-incubated | |
| | | | with human | |
| | | | gammaglobulin | |

The animals in Group I were used as a negative control group and were anesthetized with 0.1 mL of sodium pentobarbital (IMEFA), at 50 mg per kg of weight, through an IV. They were later inoculated intra-nasally with 0.5×10⁶ cells of BCG in 50 μL of Sauton solution. They were administered 25 μL/nasal cavity using an automatic micro-pipette (Plastomed), following the model for intranasal infection. The mice belonging to Group II were intra-peritoneously treated with human gammaglobulin, at 1 mg per g of weight. Group II mice were intra-nasally inoculated with 1 mg of gammaglobulin contained in 20 μL of solution. After 2 hours, the animals belonging to Groups II and III were “challenged” with 0.5×10⁶ cells of BCG administered intra-nasally. The animals of Group IV were intra-nasally inoculated with 50 μL of the BCG pre-incubated with human gammaglobulin. To achieve this, 0.5×10⁶ cells of BCG were incubated for each mg of human gammaglobulin, lightly agitated at room temperature for a period of 4 hours. The mixture was centrifuged for 10 minutes at 5585 rcf, the precipitate was re-suspended in 500 μL of PBS and was once again centrifuged in the same manner, to finally re-suspend it at a concentration of 10×10⁶ cells/mL. All of the animals were then sacrificed, at 24 hours after infection, and their right lungs were extracted in aseptic conditions for mycobiological analysis, consisting in the calculation of the number of colony-forming units per milligram of tissue (CFUs/mg of tissue).

In the case of the preparation containing IgA antibodies, this was intra-nasally administered to a group of animals, following the methodology employed in the case involving the use of the preparation of IgG antibodies, which was also utilized in the pre-incubation of BCG prior to being administered to a group of animals. Groups I and III were inoculated intra-peritoneously and intra-nasally respectively with the preparation containing IgG antibodies (Table 2). The dose of gammaglobulin utilized was the same as that used in the kinetics experiment. Following this, the animals were “challenged” with BCG. As we can see in FIG. 3, significant differences were encountered (p=0.01) upon comparing the number of CFUs recovered from the animals of Group I (negative control) with those recovered from the remaining groups. No significant differences were found between the number of CFUs recovered from Groups I, II and IV. Similar results were obtained in the group of animals that received the preparation of IgA antibodies intra-nasally and in the group of animals that were inoculated with BCG pre-incubated with this preparation. These results suggest that the interaction of antibodies specific to BCG, following their inoculation or prior to it, exercise a protecting effect which is given by the inhibition or blocking of the entry of microorganisms, by inhibiting their multiplication, as well as by their final elimination through different mechanisms. The challenge experiment demonstrated that both preparations provide similar levels of protection.

Advantages of the Proposed Solution

These kinds of preparations have the advantage of being obtained from human beings, sparing us the difficulties related to immunological incompatibility among species (diseased blood, diminished activity of the preparation due to the species' response to it, etc.) and ensuring the presence of antibodies belonging to the same species and all of the subclasses of IgG and IgA, something which guarantees that all of the immunological mechanisms dependent on the antibodies available to the species will be mobilized (neutralization, opsonization, fixing of the complement, ADCC, etc.), resulting in an optimal anti-microbial activity.

Another advantage of this kind of preparation is that it contains IgA secretory antibodies, which ensure that the penetration of the microorganism meets with a blocking or inhibiting activity. This kind of antibody is very stable and has a long half life within secretions due to the presence of the secretory component, making its anti-bacterial action all the more efficient. The presence of human lactoferrin in preparations, which appears together with the IgA secretory antibody following its purification in the colostrum, confers on it an additional anti-microbial activity due to the known, wide-ranging anti-bacterial action of lactoferrin, conditioned by its action over available iron.

Another advantage is the low level of adverse reactions, including the absence of viral transmission by specific periods of viral inactivation. Importantly, these preparations evince a prophylactic activity, exercised through a blocking of the microorganism's penetration at the level of the mucosa, and a therapeutic activity exercised through the combined action of different kinds of antibodies, which react with a large number of antigens, unleashing a great variety of anti-microbial mechanisms.

Administering the preparations through mucous membranes ensures an uncomplicated and versatile method for introducing the preparations at the site of entry of microorganisms, favoring the inhibition of infection and consequently the prophylactic effect of the preparation, as well as easy access to infected tissues (lung, intestine, etc.), also favoring the therapeutic effect exercised upon established infections.

Another of the advantages considered is the broad spectrum of activity exercised against different kinds of mycobacteria, guaranteed by the high titers of antibodies present and the broad cross reactivity of these antibodies, as well as their extraction from donors with high titer values of antibodies working against mycobacteria in their organism, something which ensures a high level of specific activity.

### BRIEF DESCRIPTION OF FIGURES

FIG. 1. Distribution kinetics of human gammaglobulin administered intra-peritoneously to Balb/c mice. The values represent the average of the titer values found in the samples extracted at each of the times of the experiment, determined through an ELISA test of complete BCG cells.

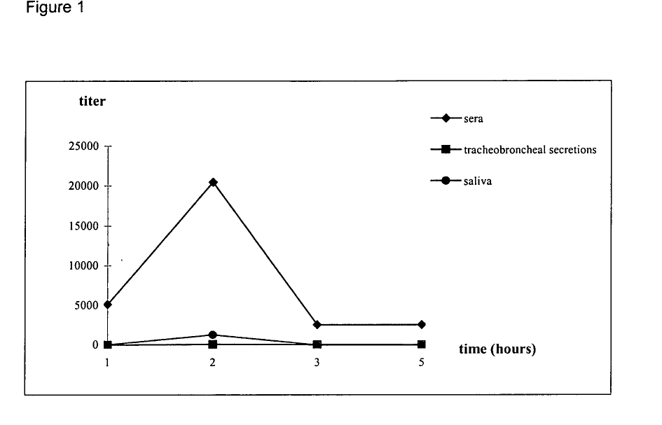

FIG. 2. Distribution kinetics of human gammaglobulin administered intra-nasally to Balb/c mice. The values represent the average titers found in the samples extracted at each of the times of the experiment, determined through an ELISA test of complete BCG cells.

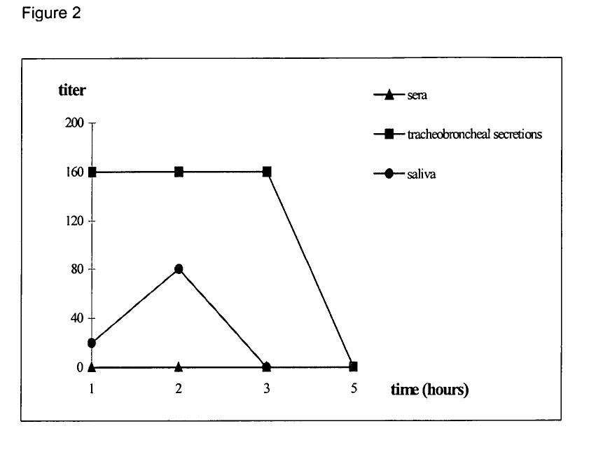

FIG. 3. Results of the challenge experiment using Balb/c mice with BCG administered intra-nasally. The values represent the average of the number of CFUs/organ calculated for each group that was studied. The animals from group 1 (positive control) were intra-nasally inoculated with BCG, those belonging to groups 2 and 3 were intra-nasally and intra-peritoneously inoculated, respectively, with human gammaglobulin, before being “challenged” with BCG administered intra-nasally. Group 4 animals were inoculated intra-nasally with BCG, pre-incubated with human gammaglobulin.

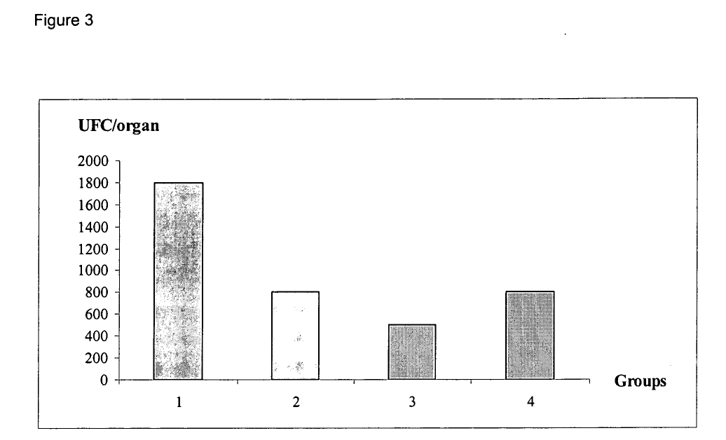

1. An anti-mycobacterial preparation containing human polyclonal antibodies of class IgG or secretory IgG or a mixture of both, active against mycobacterial components, as well as a suitable pharmaceutical medium.

2. The anti-mycobacterial preparation according to claim 1, further comprising lactoferrin.

3. The anti-mycobacterial preparation according to claim 1, wherein the human polyclonal antibodies at a concentration range of between 40 and 160 mg/ml.

4. The anti-mycobacterial preparation according to claim 2, further comprising lactoferrin at a concentration range of between 10 and 20 mg/ml.

5. The anti-mycobacterial preparation according to claim 1, wherein the mixture of both antibodies in ratios ranging from 50:50 to 80:20.

6. The anti-mycobacterial preparation according to claim 1, containing also immuno-potentiators and/or immuno-modulators.

7. The anti-mycobacterial preparation according to claim 6, further comprising immuno-potentiators and/or immuno-modulators that may be chosen from the natural, recombining, synthetic or genes group.

8. The anti-mycobacterial preparation according to claim 1, further comprising at least one combination of inactivated or attenuated strains, including those evincing heterologous antigens and/or antigens or codifying genes corresponding to said antigens.

9. A method for the use of the anti-mycobacterial preparation according to claim 1, comprising administering the preparation for an illness caused by mycobacteria.

10. A method for the use of the anti-mycobacterial preparation according to claim 1, comprising administering the preparation in combination with anti-mycobacterial drugs.

11. The method of claim 9, wherein the administering is to a human having an infection caused by mycobacteria.

12. An anti-mycobacterial preparation comprising IgA secretory antibodies.

13. The anti-mycobacterial preparation of claim 12, further comprising IgG class antibodies.

14. The anti-mycobacterial preparation of claim 13, wherein the preparation comprises a tuberculosis treatment preparation.

15. The anti-mycobacterial preparation of claim 1, further comprising lactoferrous human antibodies.
